(19) United States
(12) Patent Application Publication
(10) Pub. No.: US 2023/0058655 A1
(43) Pub. Date: Feb. 23, 2023

(54) AIR PURIFIER WITH SELF-SUSTAINING PROPERTIES

(71) Applicant: Ravi KAUSHIK, New Delhi (IN)

(72) Inventor: Ravi KAUSHIK, New Delhi (IN)

(21) Appl. No.: 17/796,818

(22) PCT Filed: Feb. 1, 2021
(86) PCT No.: PCT/IN2021/050101

§ 371 Date: Aug. 1, 2022

(30) Foreign Application Priority Data
Jan. 30, 2020 (IN) 201911049360

(51) Int. Cl. B03C 3/16; B03C 1/023; B03C 3/36; B03C 3/41; B03C 3/53; B01D 47/06; A61L 9/14

(52) U.S. Cl. CPC B03C 3/16; B03C 1/023; B03C 3/368; B03C 3/41; B03C 3/53; B01D 47/06; A61L 9/14

(57) ABSTRACT

An apparatus, and method for air purification and airborne microbial deactivation is described. In one embodiment, liquid is being used as a medium to attach air pollutants and deactivate microbes in the air. In one embodiment a negative charge is imparted for the formation of dense electric fields and negative ions using negative ion generators, corona discharge or plasma and using liquid as capturing media and disinfecting source; The air purification is done through a batch or a continuous process.

### BACKGROUND

### Technical Field

The embodiments herein relate to Air-purifiers.

### BACKGROUND OF THE INVENTION

Industrialization and power generation were the earliest causes of mass scale air pollution. The air pollution reached alarming rates in a few cities where special laws had to be enacted to overcome the hazardous conditions. The Great Smog of London in 1952 was one such event that led to enactment of the clean air act. Mexico city in the 1970s struggled with the air quality. More recently, cities such as Beijing and Delhi have been in the news for their unprecedented air pollution levels. There have been since then various laws passed in various major economies of the world to address this growing concern.

Even though law enforcement has helped curb some of the causes of pollution, the problem remains rampant. Mask has become a common wearable for the urban population even when inside home or offices.

The gaseous air pollutants of primary concern in urban settings include sulfur dioxide, nitrogen dioxide, and carbon monoxide; these are emitted directly into the air from fossil fuels such as fuel oil, gasoline, and natural gas that are burned in power plants, automobiles, and other combustion sources. Ozone (a key component of smog) is also a gaseous pollutant; it forms in the atmosphere via complex chemical reactions occurring between nitrogen dioxide and various volatile organic compounds (e.g., gasoline vapours).

Airborne suspensions of extremely small solid or liquid particles called “particulates” (e.g., soot, dust, smokes, fumes, mists), especially those less than 10 micrometres (μm; millionths of a metre) in size, are significant air pollutants because of their very harmful effects on human health. These may be emitted by various industrial processes, coal/oil burning power plants, residential heating systems, and automobiles. Lead fumes (airborne particulates less than 0.5 μm in size) are particularly toxic and are an important pollutant of many diesel fuels.

The six major air pollutants that are useful as indicators of overall air quality include but are not limited to carbon monoxide (CO), nitrogen oxides (NO and NO2), sulfur dioxide (SO2), ozone (O3), particulate matter and lead.

Various air filters are available that help curb this problem. HEPA filters are one such filters. HEPA stands for High Efficiency Particulate Air and is an efficiency standard of air filter that are able to trap 99.97 percent of particles that are 0.3 microns.

HEPA filters consist of interlaced glass fibers that are twisted and turned in myriad directions to create a fibrous maze. As particles traverse this web, they're taken out of circulation in various ways. U.S. Pat. No. 6,428,610B1 patent document discloses an HEPA filter including a multiplicity of adjacent electrostatically charged piles of nonwoven fabric. HEPA is essentially made up of non-biodegradable plastic material. If HEPA filters are continued as the main technique for air purification it may add to plastic pollution.

While using HEPA based air purifiers it is important to timely change the filters. If not removed timely, HEPA filters will introduce particulate matter into the air instead of removing it. Further, HEPA filters have a high energy requirement.

Further, the airborne disease transmission has been a big problem all around the world. The recent COVID-19 outbreak in November 2019 has further emphasized the importance of having a mechanism to purify air, especially the disinfectant with antimicrobial properties thereof.

There is therefore a need of air filters that provide air filtering while still providing easy maintenance and minimal environmental impact.

### SUMMARY

A system for purifying air is described. In one embodiment the system comprising: A chamber having an upper part and a lower part, an air inlet at the lower part of the chamber configured to let unclean air into the chamber, an air outlet at the upper part configured to let clean air exit from the chamber, a water inlet to produce a water medium within the chamber. Further, an ions producing means configured to generate charged ions to stick to air impurity present within the unclean air is disclosed. Further, two or more plates substantially parallel to each other, between the upper and lower part configured to generate an electric field to accelerate said charged ions with air impurity towards said water medium are disclosed. Further, in an embodiment, the ion producing means is configured to generate hydrogen peroxide when the ions interact with a water medium. In an embodiment, the electric field is enhanced over time with accumulation of charges on the plates.

Further, in one embodiment, at least one magnet configured to produce a magnetic field at least partially perpendicular to the electric field to accelerate charged ions with air impurities towards the water medium is available. In one exemplary embodiment, the said plates are nonconducting and parallel to the ground to hold said water medium and further comprising; a mesh of conducting wires on an upper side of the plate, said mesh connected to the ground. In an exemplary embodiment, at least one plate is conducting, parallel to the ground to hold said water medium and is connected to the ground.

In one embodiment, the water medium is a thin film of water medium on top of said plates. Further, said plates are conducting and perpendicular to the ground, with alternate plates connected to the ground. Various configurations of the plates may be possible. In one embodiment, the plates are perpendicular to the ground, with alternate plates of the said plates are conducting and connected to the ground, and remaining plates are non-conducting and not connected to the ground. In one embodiment, the water medium is a fine mist of water medium on the surface of said plates. Further, in one embodiment, a hydrogen peroxide source is configured to induce hydrogen peroxide into the chamber. This may help enhance disinfection properties.

A method for purifying air, is disclosed. The method comprising the steps of letting unclean air flow into a chamber of air purifier, generating a water medium inside the chamber, generating charged ions to stick to air impurity present within the unclean air, generating an electric field to accelerate said charged ions with air impurity towards said water medium. In one embodiment, the generation of electric field is done using two or more plates substantially parallel to each other. A further step includes causing the ions to interact with the water medium to generate hydrogen peroxide. Further, in one embodiment, the electric field is enhanced over time with accumulation of charges on the plates.

In one embodiment, the method includes: producing a magnetic field at least partially perpendicular to the electric field to accelerate charged ions with air impurities towards the water medium. Further steps include: generating an electric field by using non conducting plates, parallel to the ground to hold said water medium using a mesh of conducting wires on an upper side of the plate, said mesh connected to the ground. Further, steps include generating an electric field by using at least one plate that is conducting, parallel to the ground to hold said water medium and is connected to the ground. In one embodiment, the water medium is a thin film of water medium on top of said plate. A step discloses generating an electric field by using plates that are conducting and perpendicular to the ground, with alternate plates connected to the ground may have a variation of generating an electric field by using the plates that are perpendicular to the ground, with alternate plates of the said plates conducting and connected to the ground, and remaining plates non-conducting and not connected to the ground. In one embodiment, generating a fine mist of water medium is disclosed. In one embodiment, a step of inducing hydrogen peroxide into the chamber is disclosed. This may enhance the disinfection properties of the method acting on the unclean/polluted air.

### BRIEF DESCRIPTION OF THE DRAWINGS

These and other aspects of the embodiments herein will be better appreciated and understood when considered in conjunction with the following description and the accompanying drawings. It should be understood, however, that the following descriptions, while indicating preferred embodiments and numerous specific details thereof, are given by way of illustration and not of limitation. Many changes and modifications may be made within the scope of the embodiments herein without departing from the spirit thereof, and the embodiments herein include all such modifications.

FIG. 1 illustrates a front view of an air purifier as per an embodiment herein.

FIG. 2 illustrates an isometric blown open view of an air purifier as per an embodiment herein.

FIG. 3 illustrates a front view of an air purifier as per an embodiment herein.

FIG. 4 illustrates an isometric blown open view of an air purifier as per an embodiment herein

FIG. 5 illustrates a front view of an air purifier as per an embodiment herein.

FIG. 6 illustrates an isometric blown open view of an air purifier as per an embodiment herein.

FIG. 7 illustrates a method flow chart relating to the method of air purifying as per an embodiment herein.

### DETAILED DESCRIPTION OF THE PREFERRED EMBODIMENTS

An apparatus, and method for air purification and sanitization is described. In one embodiment, liquid is being used as a medium to attach air pollutants and deactivate pathogens using electromagnetic effect. In one embodiment, negative charge is imparted for the formation of dense electric field and negative ions using negative ion generator, corona discharge or plasma and using liquid as capturing media and disinfecting source. The air purification is done through a batch or a continuous process.

In one embodiment, permanent magnet/s could be used to induce a force to increase the drift velocity for better air purification. In another embodiment, electromagnets are used in place of permanent magnet/s.

Further in one embodiment, hydrogen peroxide is produced in the electromagnetic field where the liquid used is water. The produced hydrogen peroxide acts as a good disinfectant with antimicrobial properties. Another embodiment uses charge build-up on non-grounded plates to increase the electrostatic field for increased air purification and disinfection efficiency.

In the following description, for purpose of explanation, specific details are set forth in order to provide an understanding of the embodiments herein. It will be apparent, however, to one skilled in the art that the embodiments herein may be practiced without these details. One skilled in the art will recognize that the embodiments herein, some of which are described below, may be incorporated into a number of air purification methods and systems. Structures and devices described herein and provided in the accompanying block diagrams are illustrative of exemplary embodiments and descriptions of well-known components and processing techniques are omitted so as to not unnecessarily obscure the embodiments herein. Furthermore, connections between components within the figures are not intended to be limited to direct connections. Rather, data between these components may be modified, re-formatted, or otherwise changed by intermediary components.

Reference in the specification to “one embodiment” or “an embodiment” means that a particular feature, structure, characteristic, or function described in connection with the embodiment is included in at least one aspect of the embodiments herein. Furthermore, the appearances of the phrase “in one embodiment” in various places in the specifications are not necessarily all referring to the same embodiment.

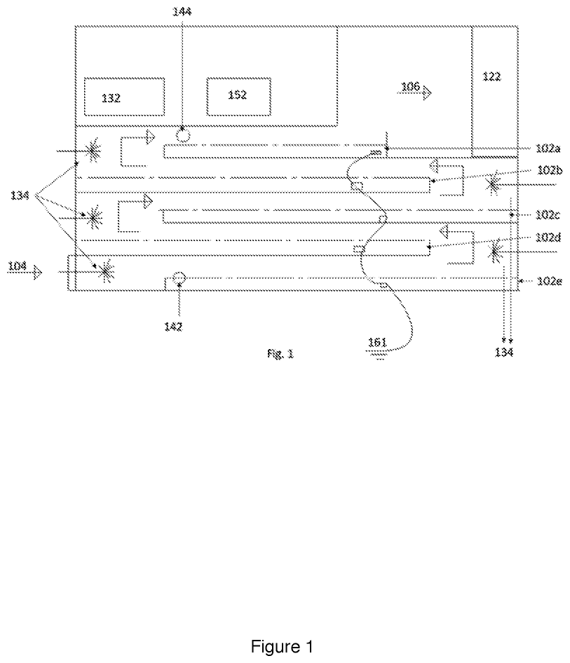

FIG. 1 illustrates a front view of the air purifier, as per an embodiment herein. In an exemplary embodiment, multiple plates 102a, 102b, 102c, 102d, 102e may be arranged in a stacked form allowing a long channel formation for the flow of air. In another embodiment, the number of the plates may vary. Further, the gap in each tray may be mathematically modeled for a particular efficiency, flow rate, voltage. For example, in one exemplary embodiment, 1 cm of air gap, 1 cm of water film depth and 9 cm of breadth may be provided. Using optical particle sizer (3330 TSI Inc.) as measuring instrument, single pass air purification efficiency for particulate matter ranging from 0.3 micrometers to 10 micrometers in number concentration units obtained may be 45-55% at 50 m³/hr flow rate. In one embodiment higher efficiency may be obtained by decreasing the flow rate (keeping every other parameters constant).

In an embodiment, a water inlet pipe may be arranged to have water poured into the top plate 102a. The plates are configured to allow flow of water from the topmost plate towards the lower plates flowing opposite to the direction of the air flow. The water may be introduced through a water inlet pipe 144 and finally exits through water outlet pipe 142. In one embodiment, a fan 122 may be placed to have flow of air out through air outlet 106. The air thus follows a path from an air inlet 104 flowing upwards. In the process of flow of polluted air over the film of water high negative electric field through ionizer nodes 134 imparted on the polluted air causes dust particles or particulate matter to get attached to the water film due to electrostatic force. Water film may be grounded by using an aluminium mesh on the plate, being connected to the ground 161. In another embodiment, any conductive and water-resistant metallic wire, mesh or plate may be used.

In yet another embodiment, alternating plates may be grounded. For example, 102a, 102c and 102e may be grounded while 102b and 102d are not grounded. The plates may be metallic or non-metallic. Care needs to be taken to have the grounded plates conducting either by virtue of being conductive (say by being metallic) or by having a thin film of water film on them.

Water due to the cohesive force and a ripple effect caused due to the passage of air at high speed over water film traps and mix the particulate matter into it. In one embodiment, liquid film can either keep continuously moving (continuous process) or it can be kept stagnant for some time and then allowed to flow (batch process) according to the availability of liquid. This may be controlled by an integrated circuit (IC) 152 and a small pump (not shown in the diagram) connected to a storage tank from where the water may be sourced.

Also, in one embodiment, unclean air being introduced can either keep continuously moving (continuous process) or it can be kept stagnant for some time and then allowed to flow (batch process).

In another embodiment, instead of using negative ionizer in the form of carbon fiber nodes, various other configurations may be provided. For example, orientation of nodes may be made against the flow of air. Better performance may be achieved when residence time of high velocity electrons can be increased. For example: orient node against the flow of air. Further the spacing among nodes may be changed to get maximum cleaning efficiency. In other exemplary embodiment, brush type node using carbon fibres may be provided. Further, output voltage of ion generator may be changed. The ionizers that may be used include but are not limited to Needle based ionizer, Wire based ionizer, Positive ions producing ionizers, Both positive and negative ion generator. In one embodiment, a conductive liquid such as water may be used. Further, addition of substances which increases the conductivity of liquids may be used to increase efficiency such as for e.g. using detergent and/or soap in water.

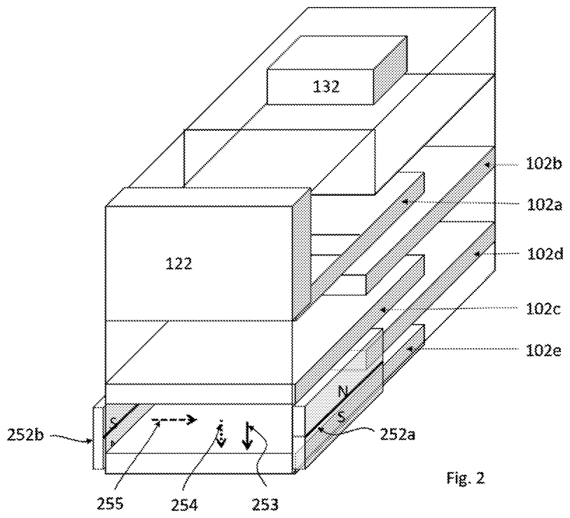

FIG. 2 illustrates an isometric blown open view of an air purifier as per an embodiment herein. This embodiment shares most features of that in reference to FIG. 1, and allows more details to be shown.

In an embodiment, instead of flowing air over the plates 102a to 102e air may be bubbled through the liquid. This may be done by channeling air to the bottom of the water film formed on the plates. In doing so some gases (such as ammonia, carbon dioxide, etc) may also be removed. Further, combination of the air flow over the film of water as well as bubbling may be used.

In yet another embodiment, variation of shape of the air purifier apparatus may be affected. For example, the plates may be made circular, or configured for better retention. Furthermore, ozone generators as well as UV lamp may be added to enhance the effect.

In one embodiment, the effect of force on air impurities stuck to the ions may be enhanced by use of magnets 252a and 252b, creating a magnetic field in the direction shown by the arrow 255. The corresponding effect on the electric field shown by the arrow 254 may be to further enhance movement of impurities attached to the negative ions, towards one of the plates in a direction shown by the arrow 253.

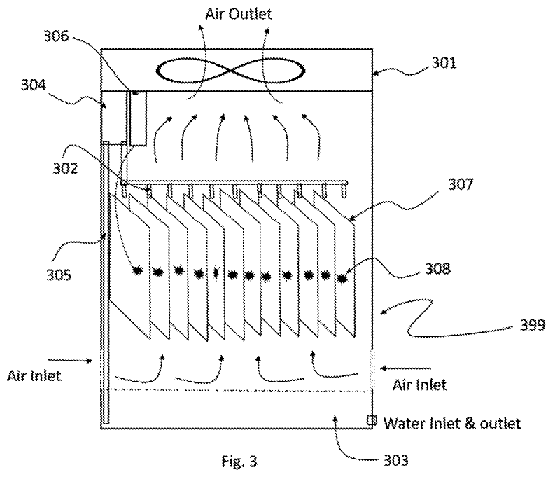

FIG. 3 illustrates a front view of an air purifier as per an embodiment herein. In an embodiment herein, plates 307 to assist in generating electromagnetic field may be placed in a vertical direction. Other embodiments, with varying angle of the plates with respect to the ground may be provided. The air may be inlet from the lower part of chamber 399. In further embodiment, as shown in FIG. 3, batch process may be used for the cleaning of the plates using pump 304 and nozzle 302 arrangements. Vertical path of air provides for lesser pressure drop. Air may be sucked from a blower 301. Combination of alternate conductive and non-conductive plates may be used to increase drift velocity of air pollutants towards the grounded plates. Also, the magnetic fields between each plate further increase the drift velocity. Pump 304, nozzle 302 and pipes 305 arrangement maybe used for continuous or periodic trickling of the liquid from the plates which gets collected in liquid tray 303. For example, in an exemplary embodiment, 1 cm of air gap can be provided between parallel plates with 30 cm as height and width. A flow rate of about 400 m³/hr will have residence time of about approx. 1 second within the parallel plate arrangement.

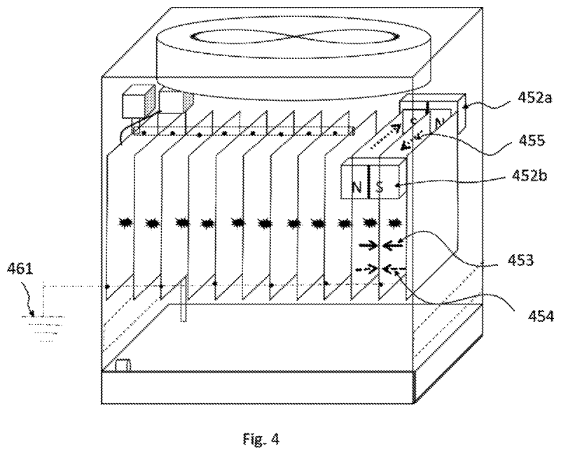

FIG. 4 illustrates an isometric blown open view of an air purifier as per an embodiment herein. This embodiment shares most features of that in reference to FIG. 3, and allows more details to be shown.

In an embodiment, the effect of force on air impurities stuck to the ions may be enhanced by use of magnets 452a and 452b, creating a magnetic field in the direction shown by the arrow 455. The corresponding effect on the electric field shown by the arrow 454 may be to further enhance movement of impurities attached to the ions, towards one of the plates in a direction shown by the arrow 453.

In an exemplary scenario, alternating plates may be grounded to ground 461. The plates may be metallic or non-metallic. Care needs to be taken to have the grounded plates conducting either by virtue of being conductive (say by being metallic) or by having a thin film of water film on them.

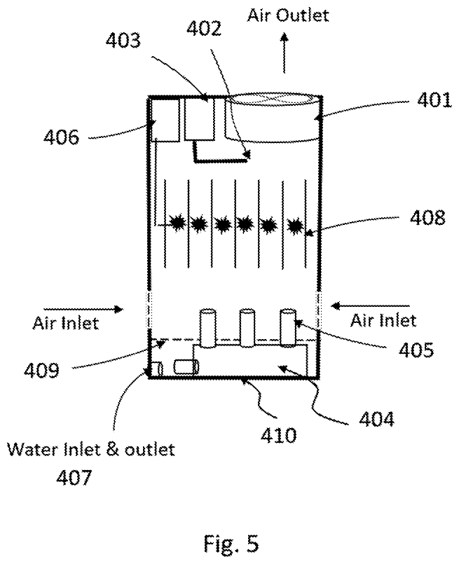

FIG. 5 illustrates a front view of an air purifier as per an embodiment herein.

In an embodiment, as shown in FIG. 5, fine liquid droplets may be formed and dispersed through the nozzle 402 using pump 403. Fine droplets may be dragged with the inflowing polluted air through the inlet using blower 401. Fine liquid droplets flowing in the direction of the flow of air increases the residence time to absorb pollutant gases from the air stream into the liquid. Airstream with fine droplets then passes through the parallel plates 408 and negative ions generator source 406 arrangement. For example, in one exemplary embodiment, 1 cm of air gap can be provided between parallel plates with 20 cm as height and width. A flow rate of about 200 m³/hr will have residence time of about 0.57 second within the parallel plate arrangement.

With time, the charge deposition on isolated or non-grounded plates will increase and make the electrostatic field denser. Particulate matter will attain negative charge though ionizers and then move towards the grounded plate due to electrostatic attraction in electrostatic fields that have been made denser, coupled with magnetic fields, ultimately leading to removal of particulate matter from the airstream. Some portion of the liquid droplets will stick to the plates and will accumulate with time until it is heavy enough to fall due to gravity in the liquid tray 410. Thus, causing a self-cleaning mechanism for the plates where heavy droplets with trapped particulate matter and absorbed gases will fall down and get collected in the liquid tray.

When fine liquid droplets such as water droplets pass through the plate and ionizer arrangement, chances of formation of hydrogen peroxide increases which aid in deactivating the microorganisms in the air. External disinfectant source 403 such as hydrogen peroxide in liquid form may be used in case of higher deactivation requirements.

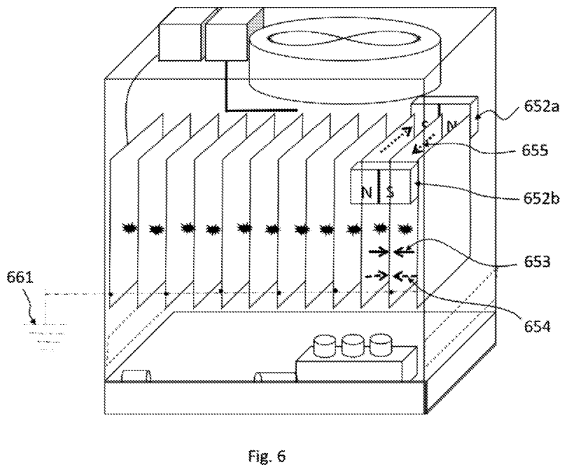

FIG. 6 illustrates an isometric blown open view of an air purifier as per an embodiment herein. This embodiment shares most features of that in reference to FIG. 5, and allows more details to be shown.

In one embodiment, the effect of force on air impurities stuck to the ions may be enhanced by use of magnets 652a and 652b, creating a magnetic field in the direction shown by the arrow 655. The corresponding effect on the electric field shown by the arrow 654 may be to further enhance movement of impurities attached to the ions, towards one of the plates in a direction shown by the arrow 653.

In an exemplary scenario, alternating plates may be grounded to ground 661. The plates may be metallic or non-metallic. Care needs to be taken to have the grounded plates conducting either by virtue of being conductive (say by being metallic) or by having a thin film of water film on them.

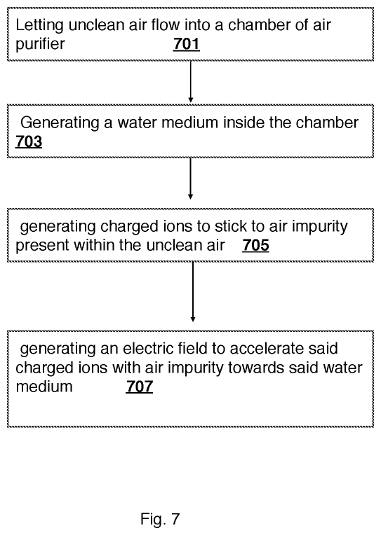

FIG. 7 illustrates a method flow chart relating to the method of air purifying as per an embodiment herein.

In one embodiment, the method comprising: letting unclean air flow into a chamber of air purifier. This may be done by means of air inlets. Further, it could be enhanced by using a blower for the flow of air. The speed/power of thrust of the fan may decide the flow of air. Further, generating a water medium inside the chamber takes place. Various other mechanisms are discussed in various parts of this disclosure.

Further step involves generating charged ions to stick to air impurity present within the unclean air. This may be done using ion generator in one embodiment.

Further, generating an electric field to accelerate said charged ions with air impurity towards said water medium takes place. It may be noted here that in an embodiment the steps maybe performed out of sequence as long as there is an overlap of each step with the other atleast partially.

Furthermore, in one embodiment the method for purifying may further comprise the step of producing a magnetic field at least partially perpendicular to the electric field to accelerate charged ions with air impurities towards the water medium. This helps in the enhanced effect of having impurities stick to the medium. Various other features as discussed in reference to the system may be present in various embodiments.

In various embodiments herein, the apparatus and method for air purification described provide for improved efficiency in terms of convenience, environmental effect and success rate. One of the reasons for the advantage may be attributed to the fact that the pollutants when ionized are made to stick to liquid film/liquid droplets, soap film rather than anything else such as wall, human skin, clothes etc as may be found in other ionization-based air purification systems. The disclosed invention deactivates the pathogens in the air effectively and can be a significant value in the future.

The air purifier as shown in various embodiments herein may be used in industries where using cooling tower water, recycled water etc may be used. Further, municipalities may use stored rainwater, groundwater etc as a water source. In cases of private organizations such as airports, colleges etc water from fountains, recycled water etc. may be used. Further, a person skilled in the art may realize that the invention may be applied to scaled up versions having high flow rates.

Furthermore, the air purifier in various embodiments may be used as car air purifier using condensed water from an air conditioner. Also, a room air purifier using condensed water from an air conditioner may be used. In case of use as a kitchen air purifier water using tap water, wastewater from dish washing, reject water from RO purifiers may be used. In yet another embodiment a pocket air purifier may be constructed.

The foregoing description of the specific embodiments will so fully reveal the general nature of the embodiments herein that others can, by applying current knowledge, readily modify and/or adapt for various applications such specific embodiments without departing from the generic concept, and, therefore, such adaptations and modifications should and are intended to be comprehended within the meaning and range of equivalents of the disclosed embodiments. It is to be understood that the phraseology or terminology employed herein is for the purpose of description and not of limitation. Therefore, while the embodiments herein have been described in terms of preferred embodiments, those skilled in the art will recognize that the embodiments herein can be practiced with modification within the spirit and scope of the description herein.

1. A system for purifying air comprising:
a. a chamber having an upper part and a lower part,
b. an air inlet at the lower part of the chamber configured to let unclean air into the chamber;
c. an air outlet at the upper part configured to let clean air exit from the chamber;
d. a water inlet to produce a water medium within the chamber
e. an ions producing means configured to generate charged ions to stick to air impurity present within the unclean air;
f. two or more plates substantially parallel to each other, between the upper and lower part configured to generate an electric field to accelerate said charged ions with air impurity towards said water medium.

2. A system for purifying air comprising, wherein the ion producing means is configured to generate hydrogen peroxide when the ions interact with water medium.

3. A system for purifying air as in claim 1, wherein the electric field is enhanced over time with accumulation of charges on the plates.

4. A system for purifying air as in claim one further comprising
a. At least one magnet configured to produce a magnetic field at least partially perpendicular to the electric field to accelerate charged ions with air impurities towards the water medium.

5. The system for purifying air as claimed in claim 1, wherein said plates are nonconducting and parallel to the ground to hold said water medium and further comprising; a mesh of conducting wires on an upper side of the plate, said mesh connected to the ground.

6. The system for purifying air as claimed in claim 1, wherein at least one plate is conducting, parallel to the ground to hold said water medium and is connected to the ground.

7. The system for purifying air as claimed in claim 3, wherein the water medium is a thin film of water medium on top of said plates.

8. The system for purifying air as claimed in claim 1, wherein said plates are conducting and perpendicular to the ground, with alternate plates connected to the ground.

9. The system for purifying air as claimed in claim 1, wherein the plates are perpendicular to the ground, with alternate plates of the said plates are conducting and connected to the ground, and remaining plates are non-conducting and not connected to the ground.

10. The system for purifying air as claimed in claim 1, wherein the water medium is a fine mist of water medium.

11. The system for purifying air as claimed in claim 1 wherein the water medium is a thin film of water medium on the surface of said plates.

12. The system for purifying air as claimed in claim 1, further comprising: a hydrogen peroxide source configured to induce hydrogen peroxide into the chamber.

13. A method for purifying air, the method comprising the steps of:
a. letting unclean air flow into a chamber of air purifier;
b. generating a water medium inside the chamber;
c. generating charged ions to stick to air impurity present within the unclean air;
d. generating an electric field to accelerate said charged ions with air impurity towards said water medium.

14. The method for purifying air, as in claim 13 wherein, generation of electric field is done using two or more plates substantially parallel to each other.

15. The method for purifying air as in claim 13, comprising the step of causing the ions to interact with the water medium to generate hydrogen peroxide.

16. The method for purifying air as in claim 13, wherein the electric field is enhanced over time with accumulation of charges on the plates.

17. The method for purifying air as in claim 13, further comprising the step of a. producing a magnetic field at least partially perpendicular to the electric field to accelerate charged ions with air impurities towards the water medium.

18. The method for purifying air as claimed in claim 13, comprising the step of generating an electric field by using non conducting plates, parallel to the ground to hold said water medium using a mesh of conducting wires on an upper side of the plate, said mesh connected to the ground.

19. The method for purifying air as claimed in claim 13, comprising the step of generating an electric field by using at least one plate that is conducting, parallel to the ground to hold said water medium and is connected to the ground.

20. The method for purifying air as claimed in claim 19, wherein the water medium is a thin film of water medium on top of said plates.

21. The method for purifying air as claimed in claim 13, comprising the step of generating an electric field by using plates that are conducting and perpendicular to the ground, with alternate plates connected to the ground.

22. The method for purifying air as claimed in claim 13, comprising the step of generating an electric field by using the plates that are perpendicular to the ground, with alternate plates of the said plates conducting and connected to the ground, and remaining plates non-conducting and not connected to the ground.

23. The method for purifying air as claimed in claim 13, comprising the step of generating a fine mist of water medium.

24. The method for purifying air as claimed in claim 13, comprising the step of generating a thin film of water medium on the surface of said plates.

25. The method for purifying air as claimed in claim 13 comprising the step of inducing hydrogen peroxide into the chamber.
